SEQUENCE LISTING (117 sequences)

SEQ ID NO: 1 - DNA, 28 bp, Artificial Sequence
  Description of Artificial Sequence Synthetic oligonucleotide
ctttccctacacgacgctcttccgatct

SEQ ID NO: 2 - DNA, 33 bp, Artificial Sequence
  Description of Artificial Sequence Synthetic oligonucleotide
ctcggcattcctgctgaaccgctcttccgatct

SEQ ID NO: 3 - DNA, 52 bp, Artificial Sequence
  Description of Artificial Sequence Synthetic primer
aatgatacggcgaccaccgagatctacactctttccctacacgacgctcttc

SEQ ID NO: 4 - DNA, 48 bp, Artificial Sequence
  Description of Artificial Sequence Synthetic primer
caagcagaagacggcatacgagatcggtctcggcattcctgctgaacc

SEQ ID NO: 5 - DNA, 24 bp, Artificial Sequence
  Description of Artificial Sequence Synthetic probe
ccctacacgacgctcttccgatct

SEQ ID NO: 6 - DNA, 24 bp, Artificial Sequence
  Description of Artificial Sequence Synthetic probe
cggcattcctgctgaaccgctctt

SEQ ID NO: 7 - DNA, 24 bp, Artificial Sequence
  Description of Artificial Sequence Synthetic primer
aatgatacggcgaccaccgagatc

SEQ ID NO: 8 - DNA, 25 bp, Artificial Sequence
  Description of Artificial Sequence Synthetic primer
caagcagaagacggcatacgagatc

SEQ ID NO: 9 - DNA, 47 bp, Artificial Sequence
  Description of Artificial Sequence Synthetic oligonucleotide
ctttccctacacgacgctcttccgatctnnnnnnagatcggaagagc

SEQ ID NO: 10 - DNA, 48 bp, Artificial Sequence
  Description of Artificial Sequence Synthetic oligonucleotide
ctcggcattcctgctgaaccgctcttccgatctnnnnnnagatcggaa

SEQ ID NO: 11 - DNA, 6 bp, Artificial Sequence
  Description of Artificial Sequence Synthetic oligonucleotide
actgat

SEQ ID NO: 12 - DNA, 6 bp, Artificial Sequence
  Description of Artificial Sequence Synthetic oligonucleotide
tagcgc

SEQ ID NO: 13 - DNA, 6 bp, Artificial Sequence
  Description of Artificial Sequence Synthetic oligonucleotide
ctattg

SEQ ID NO: 14 - DNA, 6 bp, Artificial Sequence
  Description of Artificial Sequence Synthetic oligonucleotide
accaca

SEQ ID NO: 15 - DNA, 6 bp, Artificial Sequence
  Description of Artificial Sequence Synthetic oligonucleotide
ggaatt

SEQ ID NO: 16 - DNA, 6 bp, Artificial Sequence
  Description of Artificial Sequence Synthetic oligonucleotide
caccac

SEQ ID NO: 17 - DNA, 6 bp, Artificial Sequence
  Description of Artificial Sequence Synthetic oligonucleotide
ggttgg

SEQ ID NO: 18 - DNA, 6 bp, Artificial Sequence
  Description of Artificial Sequence Synthetic oligonucleotide
ctgaca

SEQ ID NO: 19 - DNA, 6 bp, Artificial Sequence
  Description of Artificial Sequence Synthetic oligonucleotide
tacgat

SEQ ID NO: 20 - DNA, 6 bp, Artificial Sequence
  Description of Artificial Sequence Synthetic oligonucleotide
acatca

SEQ ID NO: 21 - DNA, 6 bp, Artificial Sequence
  Description of Artificial Sequence Synthetic oligonucleotide
gtggtc

SEQ ID NO: 22 - DNA, 6 bp, Artificial Sequence
  Description of Artificial Sequence Synthetic oligonucleotide
gatcgg

SEQ ID NO: 23 - DNA, 6 bp, Artificial Sequence
  Description of Artificial Sequence Synthetic oligonucleotide
tgacct

SEQ ID NO: 24 - DNA, 6 bp, Artificial Sequence
  Description of Artificial Sequence Synthetic oligonucleotide
accagg

SEQ ID NO: 25 - DNA, 6 bp, Artificial Sequence
  Description of Artificial Sequence Synthetic oligonucleotide
aggtaa

SEQ ID NO: 26 - DNA, 6 bp, Artificial Sequence
  Description of Artificial Sequence Synthetic oligonucleotide
cttatc

SEQ ID NO: 27 - DNA, 6 bp, Artificial Sequence
  Description of Artificial Sequence Synthetic oligonucleotide
tcggac

SEQ ID NO: 28 - DNA, 6 bp, Artificial Sequence
  Description of Artificial Sequence Synthetic oligonucleotide
cactta

SEQ ID NO: 29 - DNA, 6 bp, Artificial Sequence
  Description of Artificial Sequence Synthetic oligonucleotide
ggtcgt

SEQ ID NO: 30 - DNA, 6 bp, Artificial Sequence
  Description of Artificial Sequence Synthetic oligonucleotide
tgagcg

SEQ ID NO: 31 - DNA, 6 bp, Artificial Sequence
  Description of Artificial Sequence Synthetic oligonucleotide
atacca

SEQ ID NO: 32 - DNA, 6 bp, Artificial Sequence
  Description of Artificial Sequence Synthetic oligonucleotide
cactgc

SEQ ID NO: 33 - DNA, 6 bp, Artificial Sequence
  Description of Artificial Sequence Synthetic oligonucleotide
gctatt

SEQ ID NO: 34 - DNA, 6 bp, Artificial Sequence
  Description of Artificial Sequence Synthetic oligonucleotide
tagcag

SEQ ID NO: 35 - DNA, 6 bp, Artificial Sequence
  Description of Artificial Sequence Synthetic oligonucleotide
atcgct

SEQ ID NO: 36 - DNA, 6 bp, Artificial Sequence
  Description of Artificial Sequence Synthetic oligonucleotide
cctgta

SEQ ID NO: 37 - DNA, 6 bp, Artificial Sequence
  Description of Artificial Sequence Synthetic oligonucleotide
gtgaac

SEQ ID NO: 38 - DNA, 6 bp, Artificial Sequence
  Description of Artificial Sequence Synthetic oligonucleotide
tgacag

SEQ ID NO: 39 - DNA, 6 bp, Artificial Sequence
  Description of Artificial Sequence Synthetic oligonucleotide
atgtgt

SEQ ID NO: 40 - DNA, 6 bp, Artificial Sequence
  Description of Artificial Sequence Synthetic oligonucleotide
gaagtc

SEQ ID NO: 41 - DNA, 6 bp, Artificial Sequence
  Description of Artificial Sequence Synthetic oligonucleotide
tgttca

SEQ ID NO: 42 - DNA, 6 bp, Artificial Sequence
  Description of Artificial Sequence Synthetic oligonucleotide
ctcagc

SEQ ID NO: 43 - DNA, 6 bp, Artificial Sequence
  Description of Artificial Sequence Synthetic oligonucleotide
tctctg

SEQ ID NO: 44 - DNA, 6 bp, Artificial Sequence
  Description of Artificial Sequence Synthetic oligonucleotide
acatgt

SEQ ID NO: 45 - DNA, 6 bp, Artificial Sequence
  Description of Artificial Sequence Synthetic oligonucleotide
gaggca

SEQ ID NO: 46 - DNA, 6 bp, Artificial Sequence
  Description of Artificial Sequence Synthetic oligonucleotide
atcaag

SEQ ID NO: 47 - DNA, 6 bp, Artificial Sequence
  Description of Artificial Sequence Synthetic oligonucleotide
cgactc

SEQ ID NO: 48 - DNA, 6 bp, Artificial Sequence
  Description of Artificial Sequence Synthetic oligonucleotide
ccgaat

SEQ ID NO: 49 - DNA, 6 bp, Artificial Sequence
  Description of Artificial Sequence Synthetic oligonucleotide
gatgcg

SEQ ID NO: 50 - DNA, 6 bp, Artificial Sequence
  Description of Artificial Sequence Synthetic oligonucleotide
tgcagt

SEQ ID NO: 51 - DNA, 6 bp, Artificial Sequence
  Description of Artificial Sequence Synthetic oligonucleotide
tcctcc

SEQ ID NO: 52 - DNA, 6 bp, Artificial Sequence
  Description of Artificial Sequence Synthetic oligonucleotide
aggcga

SEQ ID NO: 53 - DNA, 6 bp, Artificial Sequence
  Description of Artificial Sequence Synthetic oligonucleotide
catcaa

SEQ ID NO: 54 - DNA, 6 bp, Artificial Sequence
  Description of Artificial Sequence Synthetic oligonucleotide
gcattc

SEQ ID NO: 55 - DNA, 6 bp, Artificial Sequence
  Description of Artificial Sequence Synthetic oligonucleotide
atgtag

SEQ ID NO: 56 - DNA, 6 bp, Artificial Sequence
  Description of Artificial Sequence Synthetic oligonucleotide
tgagta

SEQ ID NO: 57 - DNA, 6 bp, Artificial Sequence
  Description of Artificial Sequence Synthetic oligonucleotide
gctaag

SEQ ID NO: 58 - DNA, 6 bp, Artificial Sequence
  Description of Artificial Sequence Synthetic oligonucleotide
ctcgga

SEQ ID NO: 59 - DNA, 6 bp, Artificial Sequence
  Description of Artificial Sequence Synthetic oligonucleotide
tactct

SEQ ID NO: 60 - DNA, 6 bp, Artificial Sequence
  Description of Artificial Sequence Synthetic oligonucleotide
aggatg

SEQ ID NO: 61 - DNA, 6 bp, Artificial Sequence
  Description of Artificial Sequence Synthetic oligonucleotide
gtagcc

SEQ ID NO: 62 - DNA, 6 bp, Artificial Sequence
  Description of Artificial Sequence Synthetic oligonucleotide
tatcct

SEQ ID NO: 63 - DNA, 6 bp, Artificial Sequence
  Description of Artificial Sequence Synthetic oligonucleotide
ctaagt

SEQ ID NO: 64 - DNA, 6 bp, Artificial Sequence
  Description of Artificial Sequence Synthetic oligonucleotide
aagttg

SEQ ID NO: 65 - DNA, 6 bp, Artificial Sequence
  Description of Artificial Sequence Synthetic oligonucleotide
actcac

SEQ ID NO: 66 - DNA, 6 bp, Artificial Sequence
  Description of Artificial Sequence Synthetic oligonucleotide
gacaga

SEQ ID NO: 67 - DNA, 6 bp, Artificial Sequence
  Description of Artificial Sequence Synthetic oligonucleotide
cgttat

SEQ ID NO: 68 - DNA, 6 bp, Artificial Sequence
  Description of Artificial Sequence Synthetic oligonucleotide
agcgca

SEQ ID NO: 69 - DNA, 6 bp, Artificial Sequence
  Description of Artificial Sequence Synthetic oligonucleotide
ctgctg

SEQ ID NO: 70 - DNA, 6 bp, Artificial Sequence
  Description of Artificial Sequence Synthetic oligonucleotide
tcacgg

SEQ ID NO: 71 - DNA, 6 bp, Artificial Sequence
  Description of Artificial Sequence Synthetic oligonucleotide
gtgtcc

SEQ ID NO: 72 - DNA, 6 bp, Artificial Sequence
  Description of Artificial Sequence Synthetic oligonucleotide
catgac

SEQ ID NO: 73 - DNA, 6 bp, Artificial Sequence
  Description of Artificial Sequence Synthetic oligonucleotide
gcagct

SEQ ID NO: 74 - DNA, 6 bp, Artificial Sequence
  Description of Artificial Sequence Synthetic oligonucleotide
ggcatc

SEQ ID NO: 75 - DNA, 6 bp, Artificial Sequence
  Description of Artificial Sequence Synthetic oligonucleotide
ctatga

SEQ ID NO: 76 - DNA, 6 bp, Artificial Sequence
  Description of Artificial Sequence Synthetic oligonucleotide
tagagt

SEQ ID NO: 77 - DNA, 6 bp, Artificial Sequence
  Description of Artificial Sequence Synthetic oligonucleotide
tgtgtg

SEQ ID NO: 78 - DNA, 6 bp, Artificial Sequence
  Description of Artificial Sequence Synthetic oligonucleotide
tcacaa

SEQ ID NO: 79 - DNA, 6 bp, Artificial Sequence
  Description of Artificial Sequence Synthetic oligonucleotide
acctat

SEQ ID NO: 80 - DNA, 6 bp, Artificial Sequence
  Description of Artificial Sequence Synthetic oligonucleotide
acggtg

SEQ ID NO: 81 - DNA, 6 bp, Artificial Sequence
  Description of Artificial Sequence Synthetic oligonucleotide
cttaga

SEQ ID NO: 82 - DNA, 6 bp, Artificial Sequence
  Description of Artificial Sequence Synthetic oligonucleotide
gtcttc

SEQ ID NO: 83 - DNA, 6 bp, Artificial Sequence
  Description of Artificial Sequence Synthetic oligonucleotide
tggact

SEQ ID NO: 84 - DNA, 6 bp, Artificial Sequence
  Description of Artificial Sequence Synthetic oligonucleotide
gaacta

SEQ ID NO: 85 - DNA, 6 bp, Artificial Sequence
  Description of Artificial Sequence Synthetic oligonucleotide
agtggc

SEQ ID NO: 86 - DNA, 6 bp, Artificial Sequence
  Description of Artificial Sequence Synthetic oligonucleotide
ctctcg

SEQ ID NO: 87 - DNA, 6 bp, Artificial Sequence
  Description of Artificial Sequence Synthetic oligonucleotide
aggcat

SEQ ID NO: 88 - DNA, 6 bp, Artificial Sequence
  Description of Artificial Sequence Synthetic oligonucleotide
cctcca

SEQ ID NO: 89 - DNA, 6 bp, Artificial Sequence
  Description of Artificial Sequence Synthetic oligonucleotide
tagatc

SEQ ID NO: 90 - DNA, 6 bp, Artificial Sequence
  Description of Artificial Sequence Synthetic oligonucleotide
gaatcg

SEQ ID NO: 91 - DNA, 6 bp, Artificial Sequence
  Description of Artificial Sequence Synthetic oligonucleotide
agagat

SEQ ID NO: 92 - DNA, 6 bp, Artificial Sequence
  Description of Artificial Sequence Synthetic oligonucleotide
acttgc

SEQ ID NO: 93 - DNA, 6 bp, Artificial Sequence
  Description of Artificial Sequence Synthetic oligonucleotide
tatctc

SEQ ID NO: 94 - DNA, 6 bp, Artificial Sequence
  Description of Artificial Sequence Synthetic oligonucleotide
gccttg

SEQ ID NO: 95 - DNA, 6 bp, Artificial Sequence
  Description of Artificial Sequence Synthetic oligonucleotide
gtgcct

SEQ ID NO: 96 - DNA, 6 bp, Artificial Sequence
  Description of Artificial Sequence Synthetic oligonucleotide
ccatag

SEQ ID NO: 97 - DNA, 6 bp, Artificial Sequence
  Description of Artificial Sequence Synthetic oligonucleotide
aataat

SEQ ID NO: 98 - DNA, 6 bp, Artificial Sequence
  Description of Artificial Sequence Synthetic oligonucleotide
ggtaac

SEQ ID NO: 99 - DNA, 6 bp, Artificial Sequence
  Description of Artificial Sequence Synthetic oligonucleotide
ctatct

SEQ ID NO: 100 - DNA, 6 bp, Artificial Sequence
  Description of Artificial Sequence Synthetic oligonucleotide
gcgcta

SEQ ID NO: 101 - DNA, 6 bp, Artificial Sequence
  Description of Artificial Sequence Synthetic oligonucleotide
acgtgg

SEQ ID NO: 102 - DNA, 6 bp, Artificial Sequence
  Description of Artificial Sequence Synthetic oligonucleotide
atacac

SEQ ID NO: 103 - DNA, 6 bp, Artificial Sequence
  Description of Artificial Sequence Synthetic oligonucleotide
ggtacg

SEQ ID NO: 104 - DNA, 6 bp, Artificial Sequence
  Description of Artificial Sequence Synthetic oligonucleotide
cagtac

SEQ ID NO: 105 - DNA, 6 bp, Artificial Sequence
  Description of Artificial Sequence Synthetic oligonucleotide
tcttct

SEQ ID NO: 106 - DNA, 6 bp, Artificial Sequence
  Description of Artificial Sequence Synthetic oligonucleotide
agatac

SEQ ID NO: 107 - DNA, 68 bp, Artificial Sequence
  Description of Artificial Sequence Synthetic oligonucleotide
ctttccctacacgacgctcttccgatctnnnnnnagatcggaagagcggttcagcaggaatgccgaga

SEQ ID NO: 108 - DNA, 68 bp, Artificial Sequence
  Description of Artificial Sequence Synthetic oligonucleotide
ctcggcattcctgctgaaccgctcttccgatctnnnnnnagatcggaagagcgtcgtgtagggaaaga

SEQ ID NO: 109 - DNA, 126 bp, Artificial Sequence
  Description of Artificial Sequence Synthetic polynucleotide
aatgatacggcgaccaccgagatctacactctttccctacacgacgctcttccgatctnnnnnnagatcggaagagcggttcagcaggaatgccgagaccgatctcgtatgccgtcttctgcttga

SEQ ID NO: 110 - DNA, 126 bp, Artificial Sequence
  Description of Artificial Sequence Synthetic polynucleotide
caagcagaagacggcatacgagatcggtctcggcattcctgctgaaccgctcttccgatctnnnnnnagatcggaagagcgtcgtgtagggaaagagtgtagatctcggtggtcgccgtatcatta

SEQ ID NO: 111 - DNA, 126 bp, Artificial Sequence; identical to SEQ ID NO: 109
  Description of Artificial Sequence Synthetic polynucleotide

SEQ ID NO: 112 - DNA, 126 bp, Artificial Sequence; identical to SEQ ID NO: 110
  Description of Artificial Sequence Synthetic polynucleotide

SEQ ID NO: 113 - DNA, 59 bp, Artificial Sequence
  Description of Artificial Sequence Synthetic oligonucleotide
taatgatacggcgaccaccgagauctacactctttccctacacgacgctcttccgatct

SEQ ID NO: 114 - DNA, 126 bp, Artificial Sequence; identical to SEQ ID NO: 110
  Description of Artificial Sequence Synthetic polynucleotide

SEQ ID NO: 115 - DNA, 126 bp, Artificial Sequence; identical to SEQ ID NO: 109
  Description of Artificial Sequence Synthetic polynucleotide

SEQ ID NO: 116 - DNA, 24 bp, Artificial Sequence
  Description of Artificial Sequence Synthetic oligonucleotide
tttcaagcagaagacggcatacga

SEQ ID NO: 117 - DNA, 37 bp, Artificial Sequence
  Description of Artificial Sequence Synthetic oligonucleotide
cggtctcggcattcctgctgaaccgctcttccgatct

(19) United States
(12) Patent Application Publication
(10) Pub. No.: US 2016/0115544 A1
(43) Pub. Date: Apr. 28, 2016

(54) MOLECULAR BARCODING FOR MULTIPLEX SEQUENCING

(71) Applicant: ATHENA DIAGNOSTICS, INC., San Juan Capistrano, CA (US)

(72) Inventor: Christopher D. Elzinga, San Juan Capistrano, CA (US)

(73) Assignee: Athena Diagnostics, Inc., Worcester, MA (US)

(21) Appl. No.: 14/896,152

(22) PCT Filed: Jun. 6, 2014
(86) PCT No.: PCT/US14/41315

§ 371 Date: Dec. 4, 2015

Related U.S. Application Data
(60) Provisional application No. 61/956,170, filed on Jun. 7, 2013.

(51) Int. Cl. C12Q 1/68

(52) U.S. Cl. CPC C12Q 1/6883; C12Q 2600/158; C12Q 2600/16

(57) ABSTRACT

Described herein are methods, compositions and kits for preparing samples for multiplex next generation nucleic acid sequencing. The methods entail the use of in-line barcodes that minimize barcode-confusing chimeras, purification procedures with low cost, and/or a quantitative amplification to generate a desired amount of polynucleotides for sequencing.

### TECHNICAL FIELD

The present technology relates to low-cost sample preparation methods for multiplex next-generation sequencing (NGS).

### BACKGROUND

DNA sequencing technologies have advanced exponentially. Most recently, high-throughput sequencing (or next-generation sequencing) technologies parallelize the sequencing process, producing thousands or millions of sequences at once. In ultra-high-throughput sequencing as many as 500,000 sequencing-by-synthesis operations may be run in parallel. Next-generation sequencing lowers the costs and greatly increases the speed over the industry standard dye-terminator methods.

Massively Parallel Signature Sequencing (MPSS) was one of the earlier next-generation sequencing technologies. MPSS uses a complex approach of adapter ligation followed by adapter decoding, reading the sequence in increments of four nucleotides. This method made it susceptible to sequence-specific bias or loss of specific sequences.

Polony sequencing combined an in vitro paired-tag library with emulsion PCR, an automated microscope, and ligation-based sequencing chemistry to sequence an E. coli genome. The technology was incorporated into the Applied Biosystems SOLiD platform.

454 pyrosequencing amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs.

In Solexa sequencing DNA molecules and primers are first attached on a slide and amplified with polymerase so that local clonal colonies, initially coined “DNA colonies”, are formed. To determine the sequence, four types of reversible terminator bases (RT-bases) are added and non-incorporated nucleotides are washed away. Unlike pyrosequencing, the DNA chains are extended one nucleotide at a time and image acquisition can be performed at a delayed moment, allowing for large arrays of DNA colonies to be captured by sequential images taken from a single camera.

SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Solexa sequencing.

With the greatly reduced cost of sequencing, the cost of preparing samples, especially a large number of samples, becomes relatively high. Therefore, there is a need for an improved sample preparation method at reduced cost that is able to process multiple samples at the same time.

### SUMMARY

Described herein are methods, compositions and kits for preparing samples for multiplex next generation sequencing. The methods include the use of in-line barcodes that minimize barcode-confusing chimeras, purification procedures with low cost, and/or a quantitative amplification to generate a desired amount of polynucleotides for sequencing.

In one embodiment, the present disclosure provides a method for reducing the incidence of barcode confusing chimerism in a sample for sequencing, comprising incubating each of a plurality of samples with a first adaptor and a second adaptor, wherein: (i) each sample comprises a plurality of double-stranded target polynucleotides each having two 5′-phosphorylated blunt ends; (ii) each first adaptor is partially double-stranded comprising a first partially double-stranded fragment and a double-stranded polynucleotide barcode having a unphosphorylated blunt end, wherein all first adaptors have the same first fragment but a unique barcode, wherein neither strand of the first adaptors is longer than 40 bases, and wherein each barcode is between 6 basepairs (bp) and 8 bp long, has no more than two consecutive nucleotides being the same, and differs from any other barcode by at least 2 bp; (iii) each second adaptor is partially double-stranded having an unphosphorylated blunt end, wherein all second adaptors have the same sequence in which neither strand is longer than 40 bases, and wherein the incubation is carried out under suitable conditions for the target polynucleotides to ligate to a first adaptor at one end and to a second adaptor at the other end.

In one embodiment, provided is a method for preparing a sample for sequencing, comprising: (a) incubating each of a plurality of samples with a first adaptor and a second adaptor, wherein: (i) each sample comprises a plurality of double-stranded target polynucleotides each having two 5′-phosphorylated blunt ends; (ii) each first adaptor is partially double-stranded comprising a first partially double-stranded fragment and a double-stranded polynucleotide barcode having a unphosphorylated blunt end, wherein all first adaptors have the same first fragment but a unique barcode, wherein neither strand of the first adaptors is longer than 40 bases, and wherein each barcode is between 6 basepairs (bp) and 8 bp long, has no more than two consecutive nucleotides being the same, and differs from any other barcode by at least 2 bp; (iii) each second adaptor is partially double-stranded having an unphosphorylated blunt end, wherein all second adaptors have the same sequence in which neither strand is longer than 40 bases, and wherein the incubation is carried out under suitable conditions for the target polynucleotides to ligate to a first adaptor at one end and to a second adaptor at the other end; (b) purifying the target polynucleotides ligated with adaptors from free adaptors that are not ligated to target polynucleotides with a size-selection bead or column; (c) performing nick translation on the target polynucleotides to generate fully double-stranded polynucleotides; (d) purifying the nick translated target polynucleotides with the bead or column; (e) pooling the samples to obtain a pooled sample; (f) performing PCR amplification in the pooled sample with a first primer partially complementary to the first fragment and a second primer partially complementary to the second adaptor to obtain amplicons with sequences at both end, due to incorporation of the primers, suitable for sequencing; and (g) performing quantitative PCR (qPCR) on the amplicons to obtain a sample having a desired amount of polynucleotides for sequencing.

In some embodiments, disclosed herein is a method of detecting copy number variations in a sample of genomic DNA, comprising: i) preparing a test sample for sequencing, as recited in claim 2, ii) preparing a control sample for sequencing, as recited in claim 2, iii) performing a quantitative sequencing assay on the test sample and the control sample at a locus of interest, and iv) comparing the quantity of sequenced genomic DNA at the locus of interest in the test sample to quantify of sequenced genomic DNA at the locus of interest in the control sample, wherein deviation from the quantity of sequenced genomic DNA in the test sample as compared to the control sample is indicative of a copy number variant in the test sample.

In some aspects, the barcodes are chosen by a selection method that takes the number of samples as an input, to maximize differences between the barcodes. In some aspects, the selection method comprises generating a matrix of barcodes comprising numerical values representing the nucleotide differences between each pair of barcodes.

In some aspects, the method further comprises, prior to step (f), selecting nick translated target polynucleotides of desired regions or sequences. In some aspects, the selection comprises sequence-specific probes. In some aspects, the method further comprises amplifying the nick translated target polynucleotides with primers complementary to the first fragment and the second adaptor.

In some aspects, the method further comprises sequencing the amplicons. In some aspects, the sequencing comprises sequencing by synthesis. In some aspects, the method further comprises identifying the sequenced polynucleotide as from one of the samples by the ligated barcode sequence.

In some aspects, the purifications of the above methods are carried out with a solid-phase reversible immobilization (SPRI) bead.

In some aspects, the longer strand of the first fragment has a sequence of CTTTCCCTACACGACGCTCTTCCGATCT (SEQ ID NO: 1). In some aspects, the first primer comprises a sequence of AATGATACGGCGACCACCGAGATCTACACTCTTTCCCTACACGACGCTCTTC (SEQ ID NO: 3).

In some aspects, the longer strand of the second adaptor has a sequence of CTCGGCATTCCTGCTGAACCGCTCTTCCGATCT (SEQ ID NO: 2). In some aspects,

the second primer comprises a sequence of CAAGCAGAAGACGGCATACGAGATCGGTCTCGGCATTCCTGCTGAACC (SEQ ID NO:

4).

In some aspects, the qPCR is carried out with a first probe having a sequence of CCCTACACGACGCTCTTCCGATCT (SEQ ID NO: 5) and/or a second probe having a sequence of CGGCATTCCTGCTGAACCGCTCTT (SEQ ID NO: 6).

In some aspects, the barcodes are selected from Table 1.

In some aspects, less than about 3% amplification products are produced, during one or more PCR amplification steps of the method, from barcode confusing chimerism. In some aspects, less than about 2.5%, 2%, 1.5%, 1%, 0.5%, 0.3%, 0.2%, 0.1%, 0.05%, 0.02%, or 0.01% amplification products are produced due to barcode confusing chimerism.

Also provided, in one embodiment, is a kit comprising at least 48 polynucleotide sequences, each of which comprises a different barcode selected from Table 1. In some aspects,

the polynucleotides are partially double-stranded in which the barcodes are double-stranded having an unphosphorylated blunt end. In some aspects, the kit comprises 96 polynucleotide sequences, each of which comprises a different barcode selected from Table 1.

### BRIEF DESCRIPTION OF THE DRAWINGS

FIG. 1A-F illustrate the sample preparation and sequencing process of one embodiment of the present technology.

FIG. 2 illustrates the use of the methods disclosed herein in detection of copy number variations, wherein the method comprised a single multiplex hybridization. Genomic DNA samples were incubated with a first adaptor comprising a barcode and a second adaptor, as disclosed herein. A sample with one duplication in the DMD gene is represented by triangles. A sample with a heterozygous deletion in the DMD gene is represented by squares. A sample with a homozygous deletion in the SGCG gene is represented by diamonds. Normal samples are represented by stars (Normal 1) and circles (Normal 2). The copy number variations were clearly detectable, demonstrating expected normalized coverage, as compared to the normal samples.

### DETAILED DESCRIPTION

Described herein are primers, methods, reagents and kits for independently validating the DNA sequence of an amplicon that was, or will be, subjected to next-generation sequencing.

To facilitate an understanding of the present disclosure, a number of terms and phrases are defined below.

As used herein, unless otherwise stated, the singular forms “a,” “an,” and “the” also include the plural. Thus, for example, a reference to “an oligonucleotide” includes a plurality of oligonucleotide molecules, a reference to “a label” is a reference to one or more labels, a reference to “a probe” is a reference to one or more probes, and a reference to “a nucleic acid” is a reference to one or more polynucleotides.

As used herein, unless indicated otherwise, when referring to a numerical value, the term “about” means plus or minus 10% of the enumerated value.

The terms “amplification” or “amplify” as used herein includes methods for copying a target nucleic acid, thereby increasing the number of copies of a selected nucleic acid sequence. Amplification may be exponential or linear. A target nucleic acid may be either DNA or RNA. The sequences amplified in this manner form an “amplification product,” also known as an “amplicon.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp. 13-20; Wharam et al., Nucleic Acids Res., 29(11):E54-E54, 2001; Hafner et al., Biotechniques, 30(4):852-56, 858, 860, 2001; Zhong et al., Biotechniques, 30(4):852-6, 858, 860, 2001.

A key feature of PCR is “thermocycling” which, in the present context, comprises repeated cycling through at least three different temperatures: (1) melting/denaturation, typically at 95° C. (2) annealing of a primer to the target DNA at a temperature determined by the melting point (Tm) of the region of homology between the primer and the target and (3) extension at a temperature dependent on the polymerase, most commonly 72° C. These three temperatures are then repeated numerous times. Thermocycling protocols typically also include a first period of extended denaturation, and end on an extended period of extension.

The Tₘ of a primer varies according to the length, G+C content, and the buffer conditions, among other factors. As used herein, Tₘ refers to that in the buffer used for the reaction of interest.

As used herein, the term “detecting” refers to observing a signal from a detectable label to indicate the presence of a target. More specifically, detecting is used in the context of detecting a specific sequence.

The terms “complement,” “complementary” or “complementarity” as used herein with reference to polynucleotides (i.e., a sequence of nucleotides such as an oligonucleotide or a genomic nucleic acid) related by the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, for the sequence 5′-A-G-T-3′ is complementary to the sequence 3′-T-C-A-5′. Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. Complementarity may be “partial” in which only some of the nucleic acids' bases are matched according to the base pairing rules. Or, there may be “complete,” “total,” or “full” complementarity between the nucleic acids.

The term “detectable label” as used herein refers to a molecule or a compound or a group of molecules or a group of compounds associated with a probe and is used to identify the probe hybridized to a genomic nucleic acid or reference nucleic acid.

A “fragment” in the context of a polynucleotide refers to a sequence of nucleotide residues, either double- or single-stranded, which are at least about 2 nucleotides, at least about 5 nucleotides, at least about 10 nucleotides, at least about 20 nucleotides, at least about 25 nucleotides, at least about 30 nucleotides, at least about 40 nucleotides, at least about 50 nucleotides, at least about 100 nucleotides.

The terms “identity” and “identical” refer to a degree of identity between sequences. There may be partial identity or complete identity. A partially identical sequence is one that is less than 100% identical to another sequence. Partially identical sequences may have an overall identity of at least 70% or at least 75%, at least 80% or at least 85%, or at least 90% or at least 95%.

As used herein, the terms “isolated,” “purified” or “substantially purified” refer to molecules, such as nucleic acid, that are removed from their natural environment, isolated or separated, and are at least 60% free, preferably 75% free, and most preferably 90% free from other components with which they are naturally associated. An isolated molecule is therefore a substantially purified molecule.

The term “multiplex PCR” as used herein refers to an assay that provides for simultaneous amplification and detection of two or more products within the same reaction vessel. Each product is primed using a distinct primer pair. A multiplex reaction may further include specific probes for each product that are detectably labeled with different detectable moieties.

As used herein, the term “oligonucleotide” or “polynucleotide” refers to a short polymer composed of deoxyribonucleotides, ribonucleotides, or any combination thereof. Oligonucleotides are generally between about 10, 11, 12, 13, 14, 15, 20, 25, or 30 to about 150 nucleotides (nt) in length, more preferably about 10, 11, 12, 13, 14, 15, 20, 25, or 30 to about 70 nt.

As used herein, a “primer” is an oligonucleotide that is complementary to a target nucleotide sequence and leads to addition of nucleotides to the 3′ end of the primer in the presence of a DNA or RNA polymerase. The 3′ nucleotide of the primer should generally be identical to the target sequence at a corresponding nucleotide position for optimal extension and/or amplification. The term “primer” includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of DNA. A “reverse primer” is complementary to the sense-strand of DNA.

An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. It is a specific, i.e., non-random, interaction between two complementary polynucleotides. Hybridization and the strength of hybridization (i.e., the strength of the association between the nucleic acids) is influenced by such factors as the degree of complementary between the nucleic acids, stringency of the conditions involved, and the Tₘ of the formed hybrid.

The term “adapter” refers to a short, chemically synthesized, DNA molecule which is used to link the ends of two other DNA molecules, or to provide a common template for other manipulations, such as sequencing.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art.

As used herein, an oligonucleotide is “specific” for a nucleic acid if it is capable of hybridizing to the target of interest and not substantially hybridizing to nucleic acids which are not of interest. High levels of sequence identity are preferred and include at least 75%, at least 80%, at least 85%, at least 90%, at least 95% and more preferably at least 98% sequence identity. Sequence identity can be determined using a commercially available computer program with a default setting that employs algorithms well known in the art (e.g., BLAST).

The term “region of interest” refers to a region of a nucleic acid to be sequenced.

The term “biological sample” as used herein refers to a sample containing nucleic acids of interest. A biological sample may comprise clinical samples (i.e., obtained directly from a patient) or isolated nucleic acids and may be cellular or acellular fluids and/or tissue (e.g.,biopsy) samples. In some embodiments, a sample is obtained from a tissue or bodily fluid collected from a subject. Sample sources include, but are not limited to, sputum (processed or unprocessed), bronchial alveolar lavage (BAL), bronchial wash (BW), whole blood or isolated blood cells of any type (e.g., lymphocytes), bodily fluids, cerebrospinal fluid (CSF), urine, plasma, serum, or tissue (e.g., biopsy material). Methods of obtaining test samples and reference samples are well known to those of skill in the art and include, but are not limited to, aspirations, tissue sections, drawing of blood or other fluids, surgical or needle biopsies, collection of paraffin embedded tissue, collection of body fluids, collection of stool, and the like. In the present context the biological sample preferably is blood, serum or plasma. The term “patient sample” as used herein refers to a sample obtained from a human seeking diagnosis and/or treatment of a disease.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who possesses, or is suspected to possess, a genetic polymorphism of interest.

As used herein, the term “copy number variation” refers to alterations of DNA within a genome that result in a cell having an abnormal number of copies of one or more sections of DNA. In human genomes, copy number variants can involve homozygous or heterozygous duplications or multiplications of one or more sections of DNA, or homozygous or heterozygous deletions of one or more sections of DNA.

### Multiplexed Sample Preparation

The present disclosure provides a sample preparation method for multiplex sequencing. A multiplex sequencing can be carried out with a pooled sample that includes polynucleotides, such as genomic DNA, from multiple samples. Typically, these multiple samples contain polynucleotides of similar sequences, such as genomic DNA from different subjects for a genotyping analysis. Without proper labeling, it is difficult to identify the subject from which a particular polynucleotide is from.

Disclosed herein is a method of labeling polynucleotide samples entailing the use of polynucleotide barcodes (or simply “barcodes”) that are linked to all polynucleotide fragments from a sample. In a multiplex experiment, each sample uses a barcode that is different from other barcodes used by other samples. During the sequencing step or during post-sequencing data analysis, such barcodes can then be used to identify the source of the sequenced polynucleotides.

Misreading, i.e., incorrect identification of a base, is common among next-generation sequencing, and can be tolerated due to lack of need for high-accuracy sequence identification or overlap of sequenced fragments. Such misreading, however, can lead to misidentification of a sample when abarcode is misread. Therefore, it presents a challenge for barcode design and use.

Another problem with the use of barcodes is barcode confusing chimerism. Kircher et al., Nucleic Acid Res., 40(1):e3, 8 pages (2012) found a 0.3% contamination rate due to chimera formation during pooled PCR and sequencing, per barcode (index). Barcode contamination due to chimera formation during pooled amplification and/or pooled sequencing, a process also referred to as “jumping PCR,” is thought to occur because of template switching and recombination during PCR (Kircher et al., at page 2, left column, second paragraph). In other studies, the contamination rate was found to be as high as 5% to 7.5% (see, e.g., Li and Stoneking, Genome Biol. 13(5):R34, 15 pages (2012)). Not surprisingly, in recent versions of the GATK software, developed at the Broad Institute of MIT and Harvard, a contamination filter is used to remove such contaminates, which represent about 5% fragments in a sample.

The term “barcode confusing chimerism” is used herein in the context of PCR amplification of target polynucleotides that are ligated to adaptors that provide sequences for PCR primer binding and one or more barcode sequences for sample identification. Barcode confusing chimerism arises when multiple target polynucleotides are amplified together, as one template, to generate an amplicon in one PCR amplification due to recombination between the target polynucleotides, by virtue of their inclusion of the adaptors/barcodes. In other words, barcode confusing chimerism is the result of the sequence fragment originating from one sample being attached, during PCR amplification, to the barcode sequence assigned to (and previously ligated to the fragments of) a different sample.

The present technology provides a sample preparation method that results in no barcode confusing chimerism or a minimum level of barcode confusing chimerism, that improves cost-savings compared to other existing methods. In some aspects, less than about 3% of amplification products produced during one or more PCR amplification steps after a pool sample is prepared with the multiplex sample preparation method arise from barcode confusing chimerism. In some aspects, less than about 2.5%, 2%, 1.5%, 1%, 0.5%, 0.3%, 0.2%, 0.1%, 0.05%, 0.02%, or 0.01% amplification products are produced from such chimerism.

Fragmentation, Blunting, 5-phosphorylation, Ligation, and Nick Translation

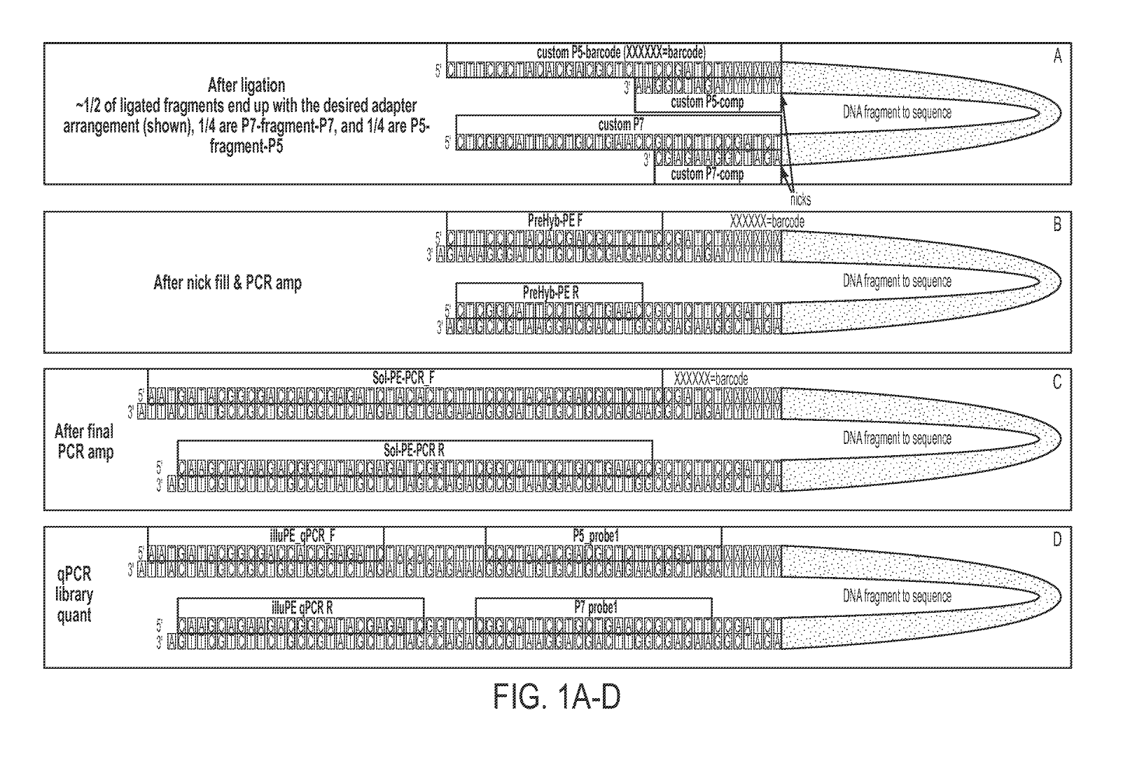

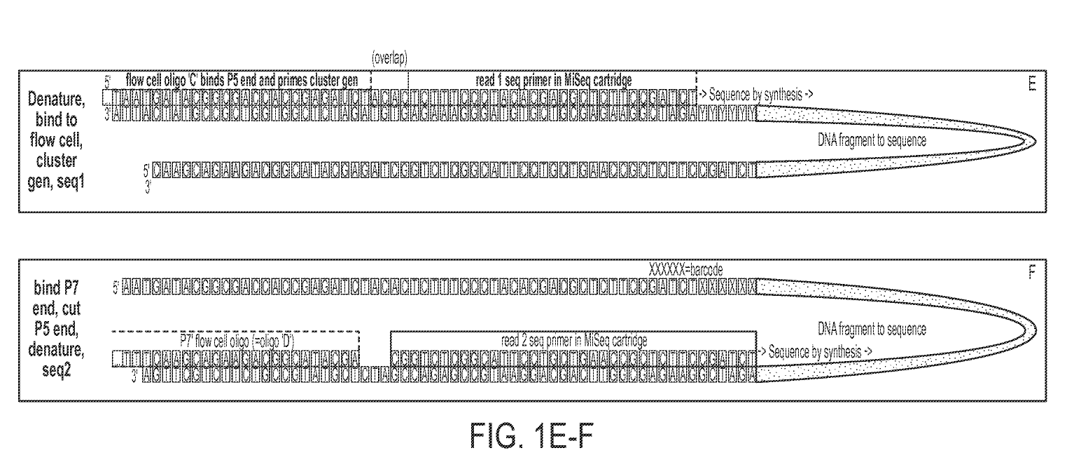

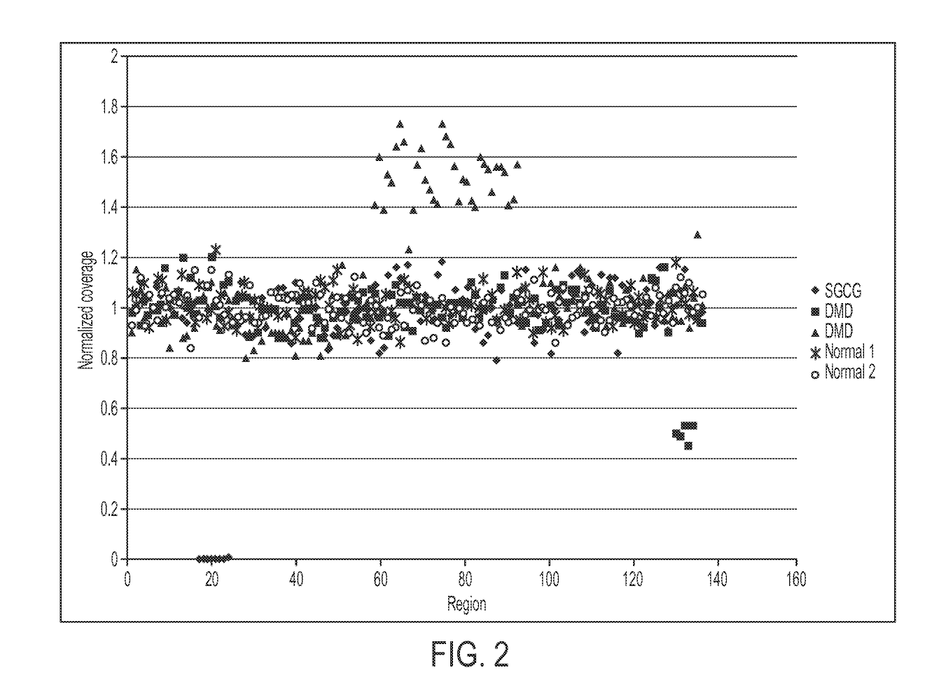

With reference to FIG. 1A, two adaptors are added to both ends of each polynucleotide fragment in a sample. Polynucleotide fragments, such as fragmented genomic DNA, can be prepared by methods known in the art, such as by sonication. With sonication, for instance, a desired average length of the fragments can be obtained by adjusting the frequency or power. In some aspects, the fragments are at least about 50 basepairs (bp) in length, or alternatively at least about 100 bp, 150 bp, or 200 bp. In some aspects, the fragments are not longer than about 1000 bp, or alternatively not longer than about 500 bp, 400 bp, 300 by or 250 bp.

Fragmented polynucleotides can then be blunted and 5′-phosphorylated so that they can be ligated to the adaptors disclosed herein. This process can be carried out with commercially available kits, such as the NEB Quick Blunting Kit®.

Both adaptors are partially double-stranded. A first adaptor (shown at the upper end of the fragment of FIG. 1A), includes a double-stranded barcode region and a partially double-stranded adaptor region (referred to as the “first fragment”). The first fragment exemplified in FIG. 1A comprises a longer strand having the sequenceCTTTCCCTACACGACGCTCTTCCGATCT (SEQ ID NO: 1), and a shorter strand that is 9-bases in length and complementary to nucleotides 20-28 of SEQ ID NO: 1.

The barcode, as exemplified in FIG. 1A, is 6-bp long (indicated as “XXXXXX”). It is understood, however, that the barcode can be longer, for instance, 7-bp or 8-bp. In some embodiments, the bar code is more than four bp long, i.e., more than 5 bp, more than 6 bp, more than 7 bp, more than 8 bp, more than 9 bp, more than 10 bp, more than 12 bp, more than 20 bp, or more than 25 bp. In some embodiments, the barcode is less than 25 bp, less than 20 bp, less than 15 bp, less than 12 bp, or less than 10 bp.Although these barcodes will be sequenced during the sequencing step, they do not create an additional large burden for sequencing.

The first fragment can include sequences useful for subsequent PCR amplification and/or sequencing. Such sequences, however, do not need to include the full length of a PCR or sequencing primer. In some aspect, the entire length (considering the longer strand) of the first adaptor is no longer than 40 bases. In some aspects, the entire length is no longer than 39 bases, or 38 bases, 37 bases, 36 bases, 35 bases, 34 bases, 33 bases, 32 bases, 31 bases or 30 bases.

Likewise, the second adaptor (lower half of FIG. 1A) is partially double-stranded, and the length of the second adaptor (considering the longer strand) is no longer than 40 bases, 39 bases, or 38 bases, 37 bases, 36 bases, 35 bases, 34 bases, 33 bases, 32 bases, 31 bases or 30 bases. The second adaptor exemplified in FIG. 1A has a longer strand having the sequence of CTCGGCATTCCTGCTGAACCGCTCTTCCGATCT (SEQ ID NO: 2), and a shorter strand that is 9-bases in length and complementary to nucleotides 21-33 of SEQ ID NO: 2.

Both adaptors are unphosphorylated at their blunt ends, so that the adaptors cannot selfligate, and can only ligate with a polynucleotide fragment, the desired sequencing target. Such ligation can be performed with methods in the art, with commercially available ligase or ligase kits.

In some aspects, the ligation is carried out with concentrations of the adaptors at a higher concentration than the polynucleotide fragments in order to reduce formation of dimers between polynucleotide fragments. In one aspect, the molar concentration of the adaptors is at least 5 times as high as that of all the polynucleotide fragments. In another aspect, the difference is at least 10 times, 20 times, 50 times, 100 times, 200 time or 1000 times.

Upon ligation, approximately half of the ligation products contain a first adaptor and a second adaptor. Approximately half contain either two first adaptors or two second adaptors. Those polynucleotides with identical adaptors at both ends, cannot be amplified or amplified efficiently, as compared to those with a first adaptor and a second adaptor in subsequent steps and tend not to interfere with the amplification and sequencing processes.

The ligation products contain two nicks (indicated in FIG. 1A) at the ligation sites, as only the polynucleotide fragments are 5′-phosphorylated prior to ligation. Further, as both ends of the ligation products have a single-stranded region that came from the adaptors, in some aspects, a subsequent nick translation step is performed to fill the nicks and extend the shorter strands. Such a procedure can be carried with, for instance, a strand displacing DNA polymerase, known in the art and commercially available.

The nick-translated polynucleotide fragments (FIG. 1B) can optionally be amplified by PCR to increase the concentration of the polynucleotide fragments, if desired.

### Barcode Design

The barcodes of the present invention have several requirements. First, a barcode used with one sample must be different from barcodes used with all other samples that will be pooled and sequenced together. Due to potential sequencing errors, larger differences are preferred so that a single-base error would not result in sample misidentification. Third, because tens of samples, or even more, are processed at one time, taking advantage of the 96-well or384-well plate formats, there are limitations on how different the barcodes can be given the short length of the barcodes.

The present disclosure provides methods to design and select barcodes to maximize the differences between barcodes in a batch.

Whether a barcode is 6, 7 or 8 bp long, there are only a finite number of possible sequences for the barcodes. With the further limitations that no more than two consecutive bases can be the same and that each barcode must be at least 2 bp different from any other barcode, the total number of sequences is further reduced. In some aspects, each barcode is at least 3 bp different from any other barcode

In some aspects, the method entails the generation of a matrix, list, or database of potential barcodes that fit the above criteria. The matrix, for instance, further includes numeral values representing the differences between barcodes. Alternatively, the barcodes are represented as binary codes or Hamming code, such that the differences are apparent. The matrix enables organization of the barcodes in a way such that a subsection (e.g., a submatrix) can be identified, having a desired number of barcodes, with maximized differences between them.

For instance, if the desired sample processing format is on a 96-well plate, a 96-member submatrix can be identified. An example list of 96 barcodes is provided in Table 1 below.

TABLE 1
An example list of barcodes that can
be used in 96 samples or fewer

| | Barcode Sequence |
|---|---|
| Well No. | (SEQ ID NO.) |
| 1 | ACTGAT (11) |
| 2 | TAGCGC (12) |
| 3 | CTATTG (13) |
| 4 | ACCACA (14) |
| 5 | GGAATT (15) |
| 6 | CACCAC (16) |
| 7 | GGTTGG (17) |
| 8 | CTGACA (18) |
| 9 | TACGAT (19) |
| 10 | ACATCA (20) |
| 11 | GTGGTC (21) |
| 12 | GATCGG (22) |
| 13 | TGACCT (23) |
| 14 | ACCAGG (24) |
| 15 | AGGTAA (25) |
| 16 | CTTATC (26) |
| 17 | TCGGAC (27) |
| 18 | CACTTA (28) |
| 19 | GGTCGT (29) |
| 20 | TGAGCG (30) |
| 21 | ATACCA (31) |
| 22 | CACTGC (32) |
| 23 | GCTATT (33) |
| 24 | TAGCAG (34) |
| 25 | ATCGCT (35) |
| 26 | CCTGTA (36) |
| 27 | GTGAAC (37) |
| 28 | TGACAG (38) |
| 29 | ATGTGT (39) |
| 30 | GAAGTC (40) |
| 31 | TGTTCA (41) |
| 32 | CTCAGC (42) |
| 33 | TCTCTG (43) |
| 34 | ACATGT (44) |
| 35 | GAGGCA (45) |
| 36 | ATCAAG (46) |
| 37 | CGACTC (47) |
| 38 | CCGAAT (48) |
| 39 | GATGCG (49) |
| 40 | TGCAGT (50) |
| 41 | TCCTCC (51) |
| 42 | AGGCGA (52) |
| 43 | CATCAA (53) |
| 44 | GCATTC (54) |
| 45 | ATGTAG (55) |
| 46 | TGAGTA (56) |
| 47 | GCTAAG (57) |
| 48 | CTCGGA (58) |
| 49 | TACTCT (59) |
| 50 | AGGATG (60) |
| 51 | GTAGCC (61) |
| 52 | TATCCT (62) |
| 53 | CTAAGT (63) |
| 54 | AAGTTG (64) |
| 55 | ACTCAC (65) |
| 56 | GACAGA (66) |
| 57 | CGTTAT (67) |
| 58 | AGCGCA (68) |
| 59 | CTGCTG (69) |
| 60 | TCACGG (70) |
| 61 | GTGTCC (71) |
| 62 | CATGAC (72) |
| 63 | GCAGCT (73) |
| 64 | GGCATC (74) |
| 65 | CTATGA (75) |
| 66 | TAGAGT (76) |
| 67 | TGTGTG (77) |
| 68 | TCACAA (78) |
| 69 | ACCTAT (79) |
| 70 | ACGGTG (80) |
| 71 | CTTAGA (81) |
| 72 | GTCTTC (82) |
| 73 | TGGACT (83) |
| 74 | GAACTA (84) |
| 75 | AGTGGC (85) |
| 76 | CTCTCG (86) |
| 77 | AGGCAT (87) |
| 78 | CCTCCA (88) |
| 79 | TAGATC (89) |
| 80 | GAATCG (90) |
| 81 | AGAGAT (91) |
| 82 | ACTTGC (92) |
| 83 | TATCTC (93) |
| 84 | GCCTTG (94) |
| 85 | GTGCCT (95) |
| 86 | CCATAG (96) |
| 87 | AATAAT (97) |
| 88 | GGTAAC (98) |
| 89 | CTATCT (99) |
| 90 | GCGCTA (100) |
| 91 | ACGTGG (101) |
| 92 | ATACAC (102) |
| 93 | GGTACG (103) |
| 94 | CAGTAC (104) |
| 95 | TCTTCT (105) |
| 96 | AGATAC (106) |

In some aspects, the method entails preparation of at least 5, 6, 7, 8, 9, 10, 20, 30, 40, 48, 50, 60, 70, 80 or 90 samples. Accordingly, a corresponding number of barcodes can be selected from the submatrix (e.g., Table 1) to suit the need.

### Cleanups

Cleanups can be performed after each step of the preceding procedure including, for instance, after polynucleotide fragmentation, after ligation, after nick translation, and/or after PCR amplification. The purpose of the cleanups is to “purify” the desired products. The term purification, as used here, does not require removal of all components in a sample that does not need to be present. Instead, a purification process enriches the concentration of the desired component in a sample relative to components intended to be removed (“contaminants”).

One purpose of the cleanups is to remove salts, buffers, nucleotides, smaller polynucleotides such as adaptors, from the samples. In this context, size selection beads or columns are contemplated to be suitable, but other methods can also be used. A size selection bead or column can separate components in a sample by virtue of their differences in molecular weight. One such example is the a solid-phase reversible immobilization (SPRI) beads, such as those sold by Agencourt Bioscience Corporation (Beverly, Mass).

In some aspects, the cleanup of each sample, after each of the steps, is carried out with the same beads or column. In other words, the beads or column used to purify the sample after ligation can be used again (“recycled”) to purify the same sample after nick translation and/or PCR amplification. This necessarily helps bring down sample preparation costs even further.

Sample pooling, selection and enrichment

The nick-translated polynucleotide fragments are ready to be pooled and analyzed, since they already contain the identifying barcodes. In some aspects, selection can be used to enrich sequences, such as particular genes or exons, that are desired to be sequenced. In some aspects, as the adaptors can be too short to include an entire sequencing primer, the polynucleotide fragments can be extended during an enrichment amplification with suitable primers (i.e., FIG. 1B to FIG. 1C).

Many methods are available for sequence selection. In one example, suitable nucleic acid probes are immobilized on a solid support as a bait to capture polynucleotide fragments having complementary sequences. In this context, it is noted that the present technology, using relatively short adaptors, helps to reduce unspecific capture of polynucleotide fragments due to binding between adaptors on different polynucleotide fragments. The selection can be performed on the pooled sample to save cost. If desired, however, the selection can be carried out for each sample individually.

In some aspects, the polynucleotide fragments in the pooled sample are subjected to PCR amplification with primers that extend the polynucleotide fragments to incorporate complete primers for subsequent amplification and/or sequencing. In some aspects, the PCR amplification employs a first primer and a second primer. The first primer contains a first portion complementary to the first fragment of the first adaptor and a second portion which, in combination with the first portion, enables subsequent sequencing. Likewise, the second primer contains a first portion complementary to the second adaptor and a second portion which, in combination with the first portion, enables subsequent sequencing.

In some embodiments, upon incorporation of these primers (see FIG. 1C), the polynucleotide fragments contain, at each end, a suitable sequence to enable next-generation sequencing on an Illumina platform. As shown in FIG. 1E-F, the additional nucleotides added to the polynucleotide fragments include a region that binds to the flow cell oligos and can be used for cluster generation, and another region for binding to a MiSeq, HiSeq, or other Illumina-compatible sequencing primer.

The example PCR product shown in FIG. 1C is amplified with a first primer of AATGATACGGCGACCACCGAGATCTACACTCTTTCCCTACACGACGCTCTTC (SEQ ID NO: 3) and a second primer of CAAGCAGAAGACGGCATACGAGATCGGTCTCGGCATTCCTGCTGAACC (SEQ ID NO: 4).

### Quantitative PCR

The pooled sample can further undergo a quantitative PCR (qPCR) amplification step which (1) further increases the concentrations of the polynucleotide fragments and (2) quantitates the amplicons such that a suitable amount of the amplicons can be collected for subsequent sequencing.

In some aspects, the qPCR uses a first probe and/or a first probe in addition to primers. Examples of such probes include CCCTACACGACGCTCTTCCGATCT (SEQ ID NO: 5, shown as P5_probe1 in FIG. 1D) and CGGCATTCCTGCTGAACCGCTCTT (SEQ ID NO: 6, shown as P7_probe1 in FIG. 1D). In some aspects, the primers used have the sequences of AATGATACGGCGACCACCGAGATC (SEQ ID NO: 7, shown as illuPE_qPCR_F in FIG. 1D) and CAAGCAGAAGACGGCATACGAGATC (SEQ ID NO: 8, shown as illuPE_qPCR_R in FIG. 1D).

In some aspects, the probes comprise one or more detectable labels. For instance, each probe can include a fluorophore (e.g., hexachlorofluorescein (HEX)) at one end. Further, such a probe can further include a quencher (e.g., IDT's Black Hole quencher 1) at the other end. The use of two probes enables greater accuracy of quantitation during qPCR.

### Sequencing and Data Analysis

After the qPCR, a desired amount of polynucleotides is used for sequencing. In some aspects, the sequencing is performed on a next-generation platform, such as Illumina's sequence-by-synthesis platform. The added sequences at both ends of the fragmented polynucleotides enable the fragments to be attached to the flow cell, at either or both ends. Sequencing can be carried out from either end on either strand(FIG. 1E-F).

From either end, the sequencing is able to identify the sequence of the polynucleotide, incorporating the barcode. The identified barcode sequence can then be used, during a post-sequencing data analysis step, to identify the fragment as corresponding to a particular sample, even when occasional misreading occurs, given the maximized differences between barcodes, as disclosed herein.

In some embodiments, the methods disclosed herein are used to detect copy number variations at a locus of interest. In some embodiments, use of the methods disclosed herein in order to detect copy number variations has the advantage of providing a single set of hybridization conditions, which minimizes background noise. In some embodiments, when detecting copy number variations, the copy number analysis is based on comparison to control samples. In some embodiments, when detecting copy number variations, the copy number analysis is based on comparison to other samples in a multiplex-run, when it can be assumed that most of the samples in a multiplex-run are normal with respect to copy number. In some embodiments, the copy number analysis comprises normalization for sample to sample variability in total sequence output. In some embodiments, to assist in the accuracy of the copy number analysis, the total sequence output in regions other than the locus of interest is also analyzed.

### Compositions and Kits

Compositions and kits suitable for carrying out the present technology are also provided. One embodiment provides compositions and kits comprising at least 48 polynucleotide sequences, each of which comprise a different barcode. In some aspects, the barcodes are selected from Table 1. In some aspects, the compositions or kits include at least 5, 6, 7, 8, 9, 10, 15, 20, 30, 40, 50, 60, 70, 80, 90, 94, 95 or 96 such polynucleotide sequences.

In some aspects, the polynucleotides are partially double-stranded, wherein the barcodes are double-stranded, having an unphosphorylated blunt end. In some aspects, the compositions or kits further include buffer, solvent, plate, and/or enzyme, as described herein, to carry out the disclosed methods.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

The disclosures illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Other embodiments are set forth within the following claims.

1. (canceled)

2. A method for preparing a sample for sequencing, comprising:
(a) incubating each of a plurality of samples with a first adaptor and a second adaptor, wherein:
(i) each sample comprises a plurality of double-stranded target polynucleotides each having two 5′-phosphorylated blunt ends;
(ii) each first adaptor is partially double-stranded comprising a first partially double-stranded fragment and a double-stranded polynucleotide barcode having a unphosphorylated blunt end, wherein all first adaptors have the same first fragment but a unique barcode, wherein neither strand of the first adaptors is longer than 40 bases, and wherein each barcode is between 6 basepairs (bp) and 8 bp long, has no more than two consecutive nucleotides being the same, and differs from any other barcode by at least 2 bp;
(iii) each second adaptor is partially double-stranded having an unphosphorylated blunt end, wherein all second adaptors have the same sequence in which neither strand is longer than 40 bases,
and wherein the incubation is carried out under suitable conditions for the target polynucleotides to ligate to a first adaptor at one end and to a second adaptor at the other end;
(b) purifying the target polynucleotides ligated with adaptors from free adaptors that are not ligated to target polynucleotides with a size-selection bead or column;
(c) performing nick translation on the target polynucleotides to generate fully double-stranded polynucleotides;
(d) purifying the nick translated target polynucleotides with the bead or column;
(e) pooling the samples to obtain a pooled sample;
(f) performing PCR amplification in the pooled sample with a first primer partially complementary to the first fragment and a second primer partially complementary to the second adaptor to obtain amplicons with sequences at both end, due to incorporation of the primers, suitable for sequencing; and
(g) performing quantitative PCR (qPCR) on the amplicons to obtain a sample having a desired amount of polynucleotides for sequencing.

3. (canceled)

4. (canceled)

5. The method of claim 2, further comprising, prior to step (f), selecting nick translated target polynucleotides of desired regions or sequences.

6. (canceled)

7. The method of claim 5, further comprising amplifying the nick translated target polynucleotides with primers complementary to the first fragment and the second adaptor.

8-10. (canceled)

11. The method of claim 2, wherein the purifications are carried out with solid-phase reversible immobilization (SPRI) beads.

12. The method of claim 2, wherein the longer strand of the first fragment has a sequence of CTTTCCCTACACGACGCTCTTCCGATCT (SEQ ID NO: 1).

13. The method of claim 12, wherein the first primer comprises a sequence of AATGATACGGCGACCACCGAGATCTACACTCTTTCCCTACACGACGCTCTTC (SEQ ID NC): 3).

14. The method of claim 2, wherein the longer strand of the second adaptor has a sequence of CTCGGCATTCCTGCTGAACCGCTCTTCCGATCT (SEQ ID NO: 2).

15. The method of claim 14, wherein the second primer comprises a sequence of CAAGCAGAAGACGGCATACGAGATCGGTCTCGGCATTCCTGCTGAACC (SEQ ID NO: 4).

16. The method of claim 2, wherein the qPCR is carried out with a first probe having a sequence of CCCTACACGACGCTCTTCCGATCT (SEQ ID NO: 5) and/or a second probe having a sequence of CGGCATTCCTGCTGAACCGCTCTT (SEQ ID NO: 6).

17. (canceled)

18. The method of claim 2, wherein less than about 3% amplification products are produced from barcode confusing chimerism.

19-21. (canceled)

22. A method of detecting copy number variations in a sample of genomic DNA, comprising
i) preparing a test sample for sequencing, as recited in claim 2,
ii) preparing a control sample for sequencing, as recited in claim 2,
iii) performing a quantitative sequencing assay on the test sample and the control sample at a locus of interest, and
iv) comparing the quantity of sequenced genomic DNA at the locus of interest in the test sample to quantify sequenced genomic DNA at the locus of interest in the control sample, wherein deviation from the quantity of sequenced genomic DNA in the test sample as compared to the control sample is indicative of a copy number variant in the test sample.

23. (canceled)

24. (canceled)

25. The method of claim 22, further comprising, prior to step (f), selecting nick translated target polynucleotides of desired regions or sequences.

26. (canceled)

27. The method of claim 25, further comprising amplifying the nick translated target polynucleotides with primers complementary to the first fragment and the second adaptor.

28-30. (canceled)

31. The method of claim 22, wherein the purifications are carried out with solid-phase reversible immobilization (SPRI) beads.

32. The method of claim 22, wherein the longer strand of the first fragment has a sequence of CTTTCCCTACACGACGCTCTTCCGATCT (SEQ ID NO: 1).

33. The method of claim 32, wherein the first primer comprises a sequence of AATGATACGGCGACCACCGAGATCTACACTCMCCCTACACGACGCTCTIC (SEQ ID NO: 3).

34. The method of claim 22, wherein the longer strand of the second adaptor has a sequence of CTCGGCATTCCTGCTGAACCGCTCTTCCGATCT (SEQ ID NO: 2).

35. The method of claim 34, wherein the second primer comprises a sequence of CAAGCAGAAGACGGCATACGAGATCGGTCTCGGCATTCCTGCTGAACC (SEQ ID NO: 4).

36. The method of claim 22, wherein the qPCR is carried out with a first probe having a sequence of CCCTACACGACGCTCTTCCGATCT (SEQ ID NO: 5) and/or a second probe having a sequence of CGGCATTCCTGCTGAACCGCTCTT (SEQ ID NO: 6).

37. (canceled)

38. The method of claim 22, wherein less than about 3% amplification products are produced from barcode confusing chimerism.
